(19) United States
(12) Patent Application Publication
(10) Pub. No.: US 2009/0203775 A1
(43) Pub. Date: Aug. 13, 2009

(54) Novel compound and a novel microorganism for producing the novel compound

(75) Inventors: Jiepeng Chen, Guangdong Province (CN); Shijin Deng, Guangdong Province (CN); Lili Duan, Guangdong Province (CN); Ronghua Wang, Guangdong Province (CN); Sulan Zhao, Guangdong Province (CN); Xuelian Qiu, Guangdong Province (CN); Shaoming Wei, Guangdong Province (CN); Peize Chen, Guangdong Province (CN); Shouxian Cheng, Guangdong Province (CN); Zhuo jun Zheng, Guangdong Province (CN); Hongmin Chen, Guangdong Province (CN); Jie Li, Guangdong Province (CN)

Correspondence Address: BIRCH STEWART KOLASCH & BIRCH, PO BOX 747, FALLS CHURCH, VA, 22040-0747, US

(73) Assignee: Haiyi Biotech Pte, Ltd.

(21) Appl. No.: 11/663,885

(22) PCT Filed: Sep. 29, 2005
(86) PCT No.: PCT/SG05/00324

§ 371 Date: Mar. 27, 2007

(51) Int. Cl. A61K 31/336; C07D 303/02; C12N 1/14; C12P 17/02; A61P 35/00; A61P 31/10; A61P 31/12

(52) U.S. Cl. 514/475; 549/545; 435/254.1; 435/123

(57) ABSTRACT

The present invention relates to a novel compound represented as by formula (I). The present invention also provides a novel strain named as Alternaria alternata var. monosporus, which can produce the compound of formula (I). The inventive strain is cultured in the medium to produce and mass the inventive compound of formula (I) in the strain and the medium. The inventive compound of formula (I) is obtained by recovering and purifying from the mycelia and medium. The compound has strong bioactivity of against cancer, fungi and viruses.

### FIELD OF THE INVENTION

The invention relates to a novel compound, and to a novel microorganism that can produce the said novel compound and a method of producing the novel compound by fermenting the microorganism. In particular, there is provided a novel strain Alternaria alternata var. monosporus.

### BACKGROUND OF THE INVENTION

Japanese laid-open Patent Application No. 5-247077 disclosed a plant growth regulator of formula (a) as described below, of which the molecular weight is 352. The plant growth regulator can be produced by shake-flask fermentation of the Cladosporium 101 in the potato extract and glucose culture medium. 73 mg of the compound can be obtained by extracting and chromatographic fractionation from the supernatant of a 7.2 L fermentation. Youji sakagami et al (Tetrahedron Letters, Vol. 36, No. 9, pp. 1469-1472, 1995) isolated an active agent named cladosporol (1) represented by formula (b) C₂₀H₁₆O₆ from the Cladosporium cladosponioides fungi, and the active agent is considered a biosynthesis inhibitor of β-1,3-glucan. Formula (a) is the horizontal structure of the formula (b).

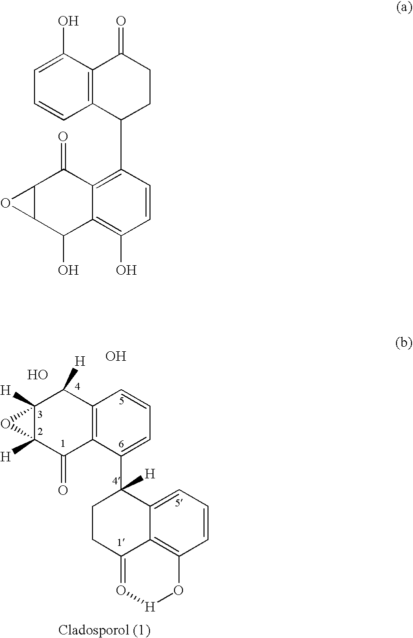

### SUMMARY OF THE INVENTION

According to a first aspect of the present invention, there is provided a novel compound represented by the following formula (I):

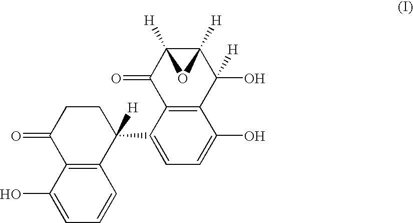

According to another aspect of the present invention, the compound (I) is produced by using a microorganism. The compound of formula (I) may also be purified and isolated from the microorganism and/or from the culture medium in which the microorganism is cultured.

Accordingly, there is also provided an isolated microorganism named as Alternaria alternata var. monosporus with the number of ST-026-R CGMCC No. 0899. In particular, the microorganism according to the invention is suitable for producing the compound of formula (I). More in particular, the microorganism is preferably under culturing conditions for producing the compound of formula (I). Accordingly, there is provided a microorganism according to the invention, wherein the microorganism is cultured in a culture medium for producing the compound of formula (I).

There is also provided a culture comprising the microorganism according to the invention, preferably in the presence of a suitable culture medium.

According to another aspect, there is provided a method for producing the compound of formula (I), comprising culturing the microorganism according to the invention in a culture medium for producing the compound of formula (I). In particular, the method further comprises the step of collecting and/or purifying the compound of formula (I).

More in particular, there is also provided a method for producing the compound of formula (I), comprising culturing the microorganism according to the invention in a culture medium to produce the compound of formula (I) in the microorganism and in the culture medium, and collecting and purifying the compound of formula (I) from the microorganism and the culture medium.

In the method, the culture medium may comprise: at least one of the following: glucose, sucrose, maltose, fructose, glycerol, starch, lactose, and galactose, any conventional material comprising a carbon source suitable for the growth of the microorganism, powder of peanut, powder of soybean, corn soup, corn steep solid, corn extract, yeast powder, peptone, beef extract, yeast extract, ammonium nitrate, ammonium chloride, powdered peanut, powdered soybean, a nitrogen source suitable for the growth of the microorganism. In particular, the ratio of the at least one carbon source and the at least one nitrogen source may be 150:1˜40:1.

The culture medium may further comprise at least one of the following: phosphate, magnesium salt, ferric salt, sodium salt, a conventional inorganic and/or organic salt suitable for the growth of the microorganism, boric acid, potassium iodide, cobalt bichloride, zinc sulfate, manganese sulfate, at least a trace element, and a conventional material comprising trace elements suitable for the growth of the microorganism.

The culture medium may further comprise at least one of the following: methyl jasmine, arachidonic acid, ammonium citrate, cerous ammonium nitrate, potassium permanganate, pyruvic acid, coumarinic acid, vanadium sulfate, α-naphthyl-acetic acid, 6-benzyl aminopurine, silver nitrate, cinnamic acid, a conventional inducer suitable for the growth of the microorganism, phenylpropyl amino acid, benzamine, sodium benzoate, sodium acetate, acetamide, propanamide, carbonic acid, ammonium acetate, and a conventional precursor suitable for the growth of the microorganism.

The culture medium may further comprise at least one inducer. The inducer may be selected from the group consisting of methyl jasmine, arachidonic acid, ammonium citrate, cerous ammonium nitrate, potassium permanganate, pyruvic acid, coumarinic acid, vanadium sulfate, α-naphthyl-acetic acid, 6-benzyl aminopurine, silver nitrate, cinnamic acid, and one or more; and/or phenylpropyl amino acid, benzamine, sodium benzoate, sodium acetate, acetamide, propanamide, carbonic acid, and ammonium acetate. The inducer may also be added while inoculate with the concentration, based on the medium, of 0.005%˜0.1%, and further a the a precursor is added as the growth of the fungus strain enters into the logarithm phase in the process of the said fermentation with the concentration, based on the medium, of 0.005%˜0.1%.

The culture medium may further comprise at least one precursor. The precursor may be selected from the group consisting of phenylpropyl amino acid, benzamine, sodium benzoate, sodium acetate, acetamide, propanamide, carbonic acid and ammonium acetate.

The culture medium may also comprises at least one of the following: one or more materials providing a carbon source selected from the group consisting of glucose, sucrose, maltose, fructose, glycerol, starch, lactose and galactose; one or more materials providing a nitrogen source selected from the group consisting of the powdered peanut, powdered soybean, corn extract, powdered yeast, peptone, beef extract, yeast extract, ammonium nitrate, and ammonium chloride; one or more inducers selected from the group consisting of methyl jasmine, arachidonic acid, ammonium citrate, cerous ammonium nitrate, potassium permanganate, pyruvic acid, coumarinic acid, vanadium sulfate, α-naphthyl-acetic acid, 6-benzyl aminopurine, silver nitrate and cinnamic acid; and one or more precursors selected from the group consisting of phenylpropyl amino acid, benzamine, sodium benzoate, sodium acetate, acetamide, propanamide, carbonic acid and ammonium acetate.

In the method according to the invention, the microorganism may be cultured in an aerobic condition at temperature of 23˜29° C., wherein the initial pH of the fermentation is 5.5˜11.0; the pH is adjusted to 6.0˜7.5 in the metaphase and anaphase.

The method according to the invention may comprise a step of collecting and/or purifying the compound of formula (I). In particular, the collecting and/or purifying the compound of formula (I) comprises the following steps of:
(a) isolating the cultured microorganism and/or supernatant obtained from the culture medium;
(b) extracting the cultured microorganism with a first organic solution to obtain a first organic extract, and/or extracting the supernatant with a second organic solution to obtain a second organic extract, and drying the first and/or second organic extracts to obtain a product;
(c) purifying the product obtained from the step (b) by at least one chromatography process and at least one crystallization process to obtain a substantially purified compound of formula (I).

The at least chromatography process may comprise the following conditions: at least one chromatographic column is used; stationary phase in the column of the chromatography comprises silica gel or alumina; and

the mobile phase comprises at least one of the following solvent systems selected from the group consisting of ethane/ethyl acetate; methanol/methylene chloride; ethanol/methylene chloride; and acetone/methylene chloride.

The at least one crystallization process may comprise:

dissolving the product in an alcohol solution,
adding water;
reducing temperature to 4˜10° C. for 1˜12 hours to separate out the compound of formula (I) in crystallized form;
filtering the compound; and
drying the compound
wherein the at least one crystallization process may be repeated to obtain substantially purified compound of formula (I).

In particular, the at least one crystallization process may comprise:

dissolving the products in ethyl acetate solution;
adding petroleum ether;
cooling to 4˜10° C. to crystallize the compound of formula (I);
filtering the compound; and
drying the compound
wherein the at least one crystallization process may be repeated to obtain substantially purified compound of formula (I).

In the method according to one aspect of the invention, before the step (a), the pH of the culture solution may be adjusted to pH 2˜9.

The microorganism may be dried and crushed before step (b).

According to another aspect, there is provide a compound (I) for use in medicine. For example, for use as anticancer, antifungal, and/or antiviral.

There is also provided an anticancer medicament comprising the compound of formula (I). There is also provided an antifungal medicament comprising the compound of formula (I). There is also provided an antiviral medicament comprising the compound of formula (I). The medicament may further comprise a pharmaceutically acceptable excipient, diluent and/or carrier.

There is also provided a pharmaceutical composition comprising the compound of formula (I). The pharmaceutical composition may further comprise a pharmaceutically acceptable excipient, diluent and/or carrier.

There is also provided a use of the compound of formula (I) in the manufacture of a medicament for the treatment of a cancer, fungi, and/or viruses.

There is also provided a kit of parts for treating a disease comprising the compound (I), optionally with information related to the use of the compound included in the kit. In particular, the kit is for the treatment of cancer, fungi, and/or viruses.

According to another aspect, there is provided a method of treating cancer comprising administering to an animal an effective amount of the compound (I). There is also provided a method of treating fungi comprising applying to an animal an effective amount of the compound (I). There is also provided a method of treating a viral infection comprising administering to an animal an effective amount of the compound (I). The animal may be a mammal. The mammal may be human or a non-human mammal.

### BRIEF DESCRIPTION OF THE DRAWINGS

FIG. 1 and FIG. 2 show the spore of the inventive novel fungal strain.

FIG. 3 shows the hypha of the inventive novel fungal strain.

FIG. 4 shows the TLC graph of the sample extracted from the fermented mycelia by ethyl acetate.

FIG. 5 shows the HPLC Spectrum of the compound of formula (I).

FIG. 6 shows the UV Spectrum of the compound of formula (I) produced by the inventive method.

FIG. 7 shows the IR Spectrum of the compound of formula (I) produced by the inventive method.

FIG. 8 shows the FAB-MS Spectrum of the compound of formula (I) produced by the inventive method.

FIG. 9 shows a series of NMR Spectrum (including ¹H-NMR, FIG. 9A; ¹³C-NMR, FIG. 9B; and ¹H-¹H COSY Spectrum, FIG. 9C) of the compound of formula (I) produced by the inventive method.

FIG. 10 shows the X-ray Diffraction Spectrum of the compound of formula (I) produced by the inventive method.

FIG. 11 shows the curve of the percentage of killed cells by using the inventive compound of formula (I) in different cells.

FIG. 12 shows the curve of inhibitory effect of the inventive compound of formula (I) on the growth of the transplanted human stomach cancer in the MGC-803 nude mouse.

FIG. 13 show the chemical structure of the compound of formula (I).

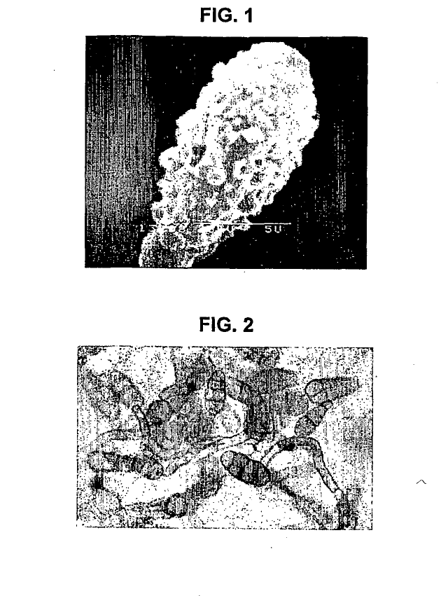

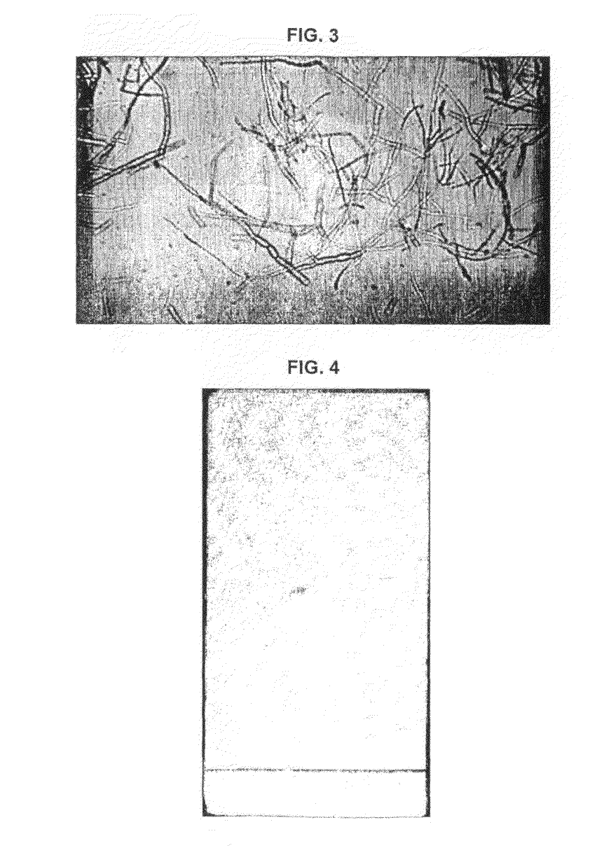

### DETAILED DESCRIPTION OF THE INVENTION

The object of the present invention is to provide a novel and a pharmaceutically effective compound.

The object of the present invention is also to provide a novel microorganism strain, which can produce the said compound.

The object of the present invention also provides a method of producing the said compound by fermenting the said microorganism strain.

Through many years of deep and careful researches, the inventors have surprisingly found, cultured and induced a novel microorganism strain, which can produce a novel compound. Through lots of experimental researches, the inventors have also invented a method of producing the said novel compound by fermenting the said microorganism strain.

The present invention provides a novel compound having the following formula (I):

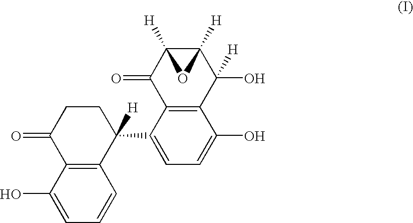

### Color: White Crystal

Melting point: 187˜191° C.
Specific rotation: 213.01° (Acetone solution 1.230 mg/ml)
Molecular weight: 352
Molecular formula: C₂₀H₁₆O₆
UV λₘₐₓnm (ε): 212 nm (FIG. 6)

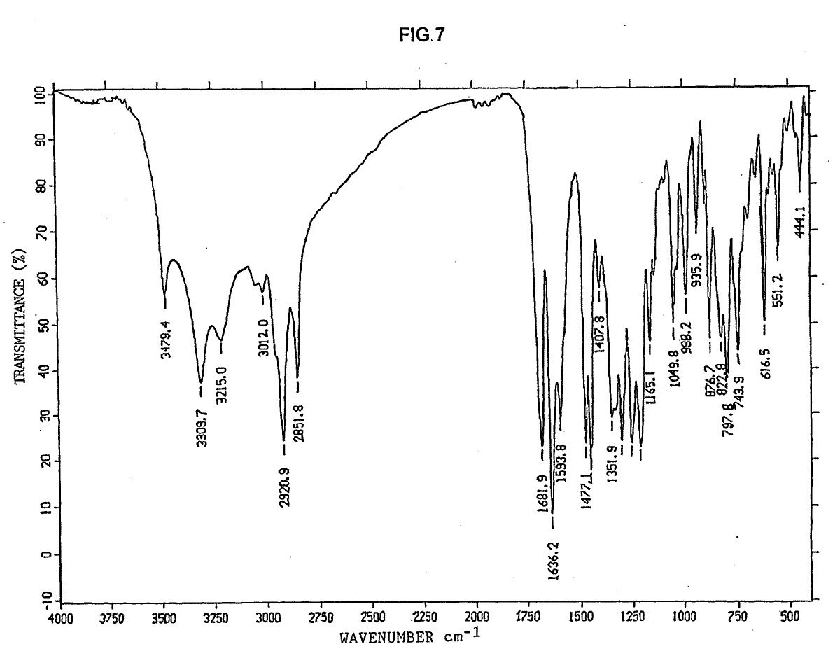

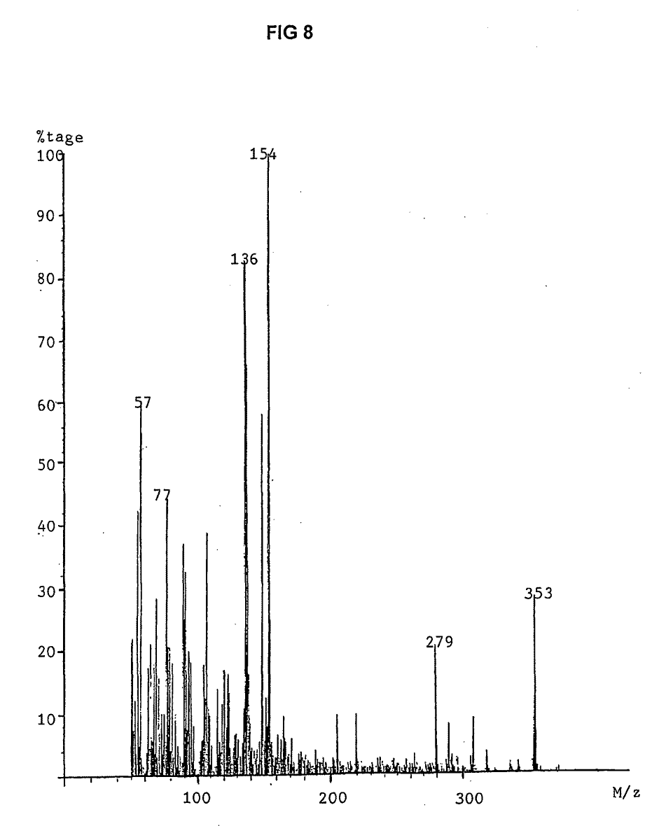

### IR Spectrum: (FIG. 7)

### MS Spectrum: (FIG. 8)

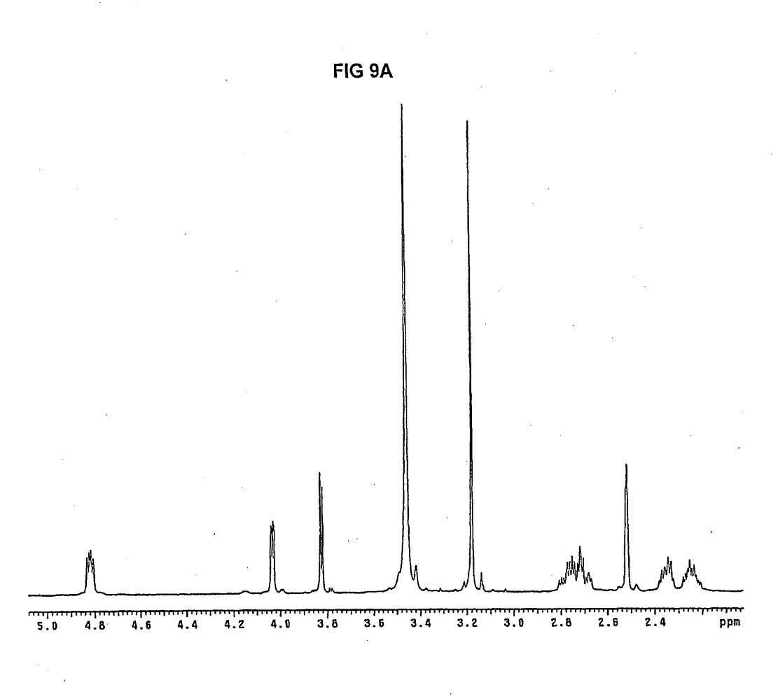

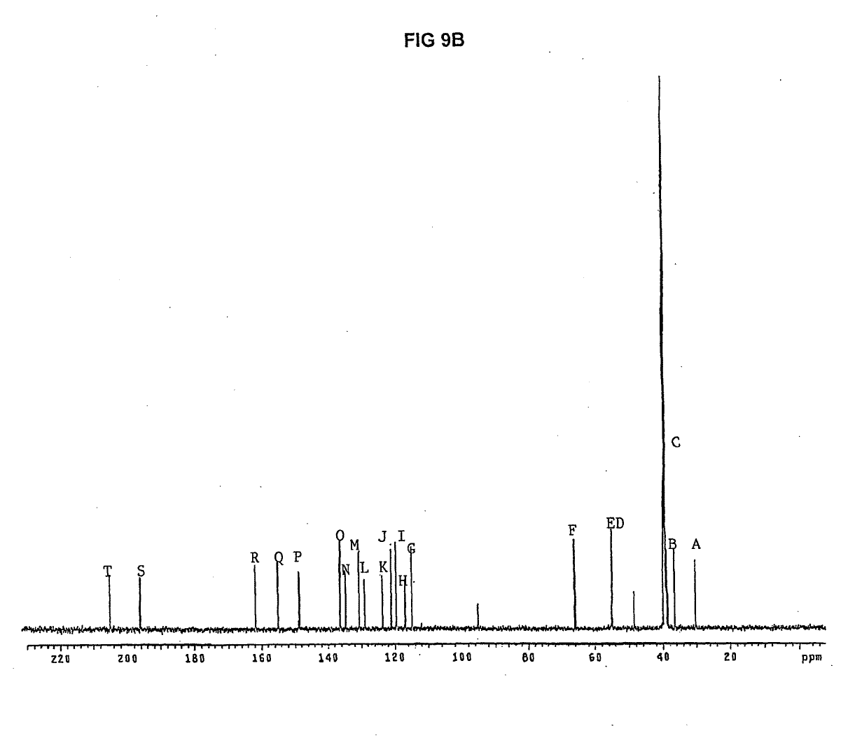

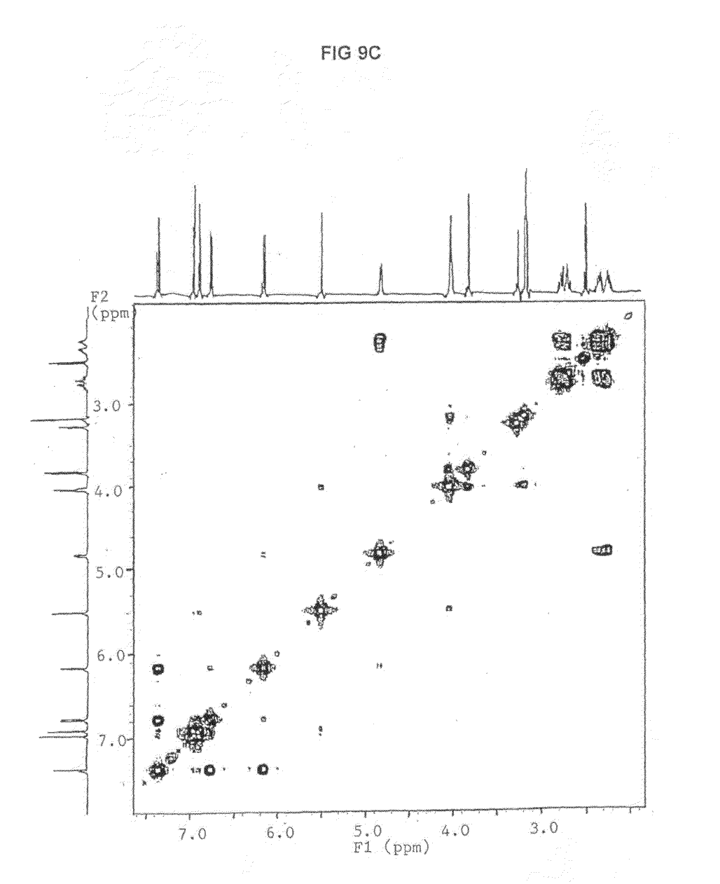

### NMR Spectrum: (FIG. 9)

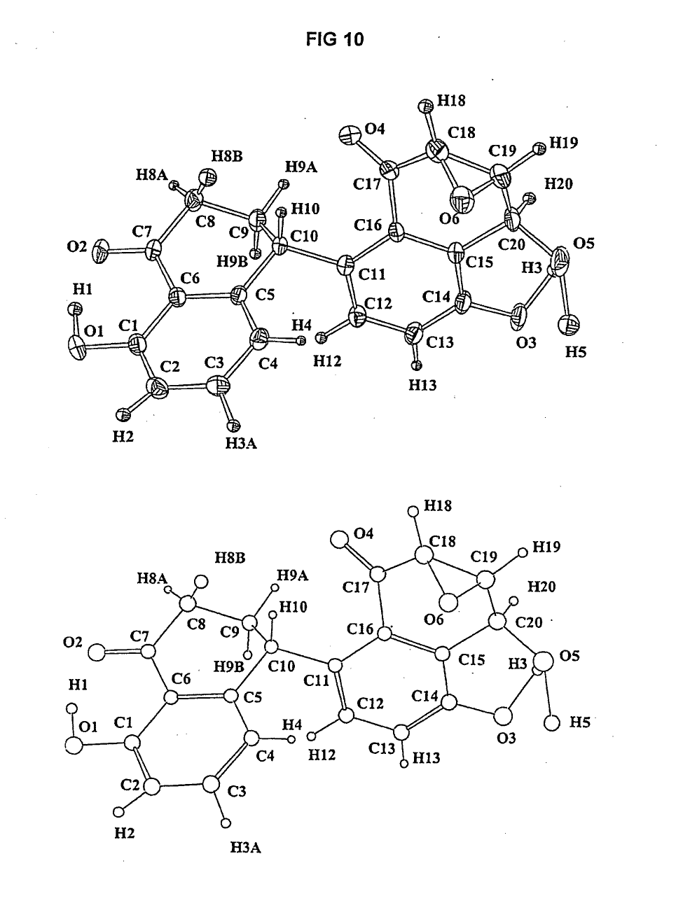

### X-ray Diffraction Spectrum: (FIG. 10)

The three-dimensional structure of the novel compound of the invention is different from that of the well-known compound of previously described formula (b). The epoxy of the inventive compound is extraversion, while the epoxy of well-known compound of previously described formula (b) is introversion. The inventive compound has specific and surprising bioactivity due to the differences in its three-dimensional structure. The compound of the present invention has extremely strong bioactivity against various cancers such as stomach cancer, leukemia, breast cancer and ovary cancer and the like. Meanwhile, the substance also has high anti-fungi and anti-viral bioactivity. Therefore the compound can be explored as a new generation of anti-cancer, anti-fungi and anti-virus medicament. The range of potential applications of the compound is extensive.

The novel microorganism strain provided by the present invention which can produce the compound presented as formula (I) is an Alternaria alternata var. monosporus ST-026R with deposit number CGMCC No. 0899.

The inventors collected 310 samples from the branches, barks, leaves and roots of seven over 300-year-old Taxus yunnanensis growing in the conifer and broad-leaf mixed forest at the 2500˜3000 meter zone above sea level of the LaoJun Mountain in Lijiang Region of Yunnan Province, China. Under sterile conditions, the sample pieces were sterilized with 60% ethanol, washed by sterile water, dried by aspirating the excess water and then the tissue was torn into many thin slices layer by layer. The samples were cut into little parts, arranged in the order of from the inter tissue layers to the outer layers, and then the samples were inoculated into liquid agar medium, and cultured at 25° C. for 7˜15 days. Various kinds of microorganisms grew up one after another. Fermentations were performed after the strains of different form, different color were purified. Strains with different morphology and colours were cultured and isolated. The strain having high yield ratio of producing both the inventive compound of the present invention and taxol was selected as the initial strain and induced by ultraviolet radiation. After generations of inductions by UV irradiation, a mutant fungal strain with morphological variations was obtained. The monosporus fungal strain thus obtained produces the inventive novel compound. This fungal strain inheriting this characteristic was then used as an initial strain for subsequent cultures. The variant strain described above was cultured for many generations until the mutant strain was finally stable to obtain the inventive novel monosporus strain. According to plant nomenclature convention, the novel fungal strain was named Alternaria alternata var. monosporus with the code number of ST-026-R. The strain is deposited in China General Microbiological Culture Collection Center (CGMCC), and its address is No. 13, Beiyitiao, Zhongguancun, Institute of Microbiology, Chinese Academy of Sciences, Beijing, China. The depositing number is CGMCC 0899.

The novel strain of CGMCC 0899 provided by the present invention has the following microorganism characters:

The length of the hypha cell is approximately equal to the width or the length is approximately two times than the width. The middle of the cell is narrowed and the two extreme parts of the cell are swollen. The horizontal compartment is also narrowed, and the whole hypha looks like a chain belt. The branch of the hypha is short, and the angle of the branches is acute. The chain of the conidiophore is short, having two branches. Most of the spores are unicellular, colorless and transparent, without wart. The size of the spore is around 20-25 μm in shape of olivary or clavated, and the trace of the spore is clear.

On the culture dish with potato dextrose agar (PDA), the inventive strain is extended, round, with high density, and having two rings. The edge of the strain is clear and gray, the inner ring of the strain is black. The hypha is colorless or olive colored, having branches and horizontal compartments. There are two kinds of hyphae: thick and thin hypha. The growth of the hypha reaches to saturated state with large amount of spores on a 90 mm dish in 5 days due to its fast growing characteristic. The hyphae form into wood-like blocks. Young hyphae always form into single or bunchy transparent, cystiform or moniliform spore, which is two times thicker than the hypha. The spore can bourgeon into new hypha. The top of the hypha shows a sharp, thin and transparent long cell.

The present strain produces three kinds of spores, the first is the conidiophore. The sporular chains grow from the top of the branches of the hypha, each top of the branch can form sporular chain. Commonly, the sporular chain consists of 4˜6 sporules, some sporular chains consist of 8˜10 sporules. Most of the peduncles of the conidiophore are columniform, transparent, single and perpendicular, comprising four cells. The young conidiophore in dark shows olivary or ellipse with lots of granules on the surface. The compartment is not clear. Young conidiophores are approximately 21˜25×11˜15 μm in size. The width of the hypha: is 4 μm, and the length is 2.0 μm. The sporular chain can also be formed from the top and the side of the sporule. The adult conidiophore is olivary with light color in the size of 25˜15 μm, having 4˜8 horizontal compartments and 1˜2 vertical compartments. The intercellular space is narrowed, and the length is 2.0 μm.

The second kind of spore is the brood cell, which is produced during the early stage of the growth of the hyphae and in the liquid culture medium. It is the specific sporule that belongs to the endogenous fungi, cystiform and round. It can be bourgeoned into the hypha from the side. The third one is disclosed for the first time, and has not been reported.

The third one has two hyphae that are parallel, producing a jointed tube. A transparent, light brown elliptic sporule is produced in the middle part of the jointed tube. The adult sporule has 4˜6 horizontal compartments, 2˜3 vertical compartments. The development of the sporule is conjugative reproduction, just similar to that of alga, but not the sexual reproduction.

The inventive novel strain No. CGMCC 0899 can not only produce the inventive compound, but also can also produce the anti-cancer medicament, taxol.

The present invention provides a method of producing the inventive compound of (1) by using the inventive novel strain, CGMCC0899. The said method comprises culturing or fermenting the inventive strain CGMCC 0899 in the culture medium in order to produce and amass the inventive compound of (I) in the said strain and in the said culture or fermentation medium, and collecting and purifying the novel compound of (I) from the said strain and the medium. In the present description, the terms “culturing” and “fermenting” both refer to growing the fungal strain under non-natural conditions and the terms are used interchangeably. The terms “culture” and “fermnentation” are construed accordingly.

The said culture medium can be any one of conventional and well-known culture media in the art of mycology. However, conventional culture media are not optimal. The preferred culture media have materials or components different from conventional culture media. Also, specific culture conditions, inoculation and culture techniques used in the method of the present invention enable the microorganism of the present invention to produce surprisingly high yield ratios of the compound of formula (I). The preferred culture medium may have carbon and nitrogen sources provided. Other organic or inorganic materials can be added into the culture medium to promote the growth of the microorganism and to improve the yield ratio of the inventive novel compound of formula (I). The said carbon sources include, but are not limited to, glucose, sucrose, maltose, fructose, glycerol, starch, lactose and galactose. The useful nitrogen sources include, but are not limited to, powdered not limit to, the powder of the peanut, the powder of the soybean, the corn soup, the powder of the yeast, the protein, protein, peptone, beef extract, yeast extract, ammonium nitrate and ammonium chloride. Preferably, the ratio of the carbon source and the nitrogen source is 150:1˜40:1. The said inorganic materials include, but not limit to, phosphate, magnesium salt, for example bitter salt, iron salt, for example ferrous sulfide, iron trichloride, sodium salt, for example potassium sodium tartrate. The said culture medium includes, but not limit to, trace elements, for example boric acid, potassium iodide, cobalt bichloride, zinc sulfate, manganese sulfate, inducer, for example methyl jasmine, arachidonic acid, ammonium citrate, cerous ammonium nitrate, potassium permanganate, pyruvic acid, coumarinic acid, vanadium sulfate, α-naphthyl-acetic acid, 6-benzyl aminopurine, silver nitrate, cinnamic acid etc, precursor, for example, phenylpropyl amino acid, benzamine, sodium benzoate, sodium acetate, acetamide, propanamide, carbonic acid, ammonium acetate and the like.

The said culture can be performed under the conventional or well-known condition in the art. The said fermentation can be performed under aerobic conditions at 23˜29° C. The initial pH can be 5.5˜11.0, preferably 6.0˜7.0, in the metaphase and anaphase, the pH can be regulated to 6.0˜7.5. The duration of culture is dependent on the culture condition, for example, the fermentation can be performed for 6˜9 days. During the process of the said fermentation, preferably, the inducer, such as methyl jasmine, arachidonic acid, ammonium citrate, cerous ammonium nitrate and potassium permanganate, is added while inoculating. The preferable concentration is (versus culture medium) 0.005˜0.1%. During the process of the said fermentation, as the growth of the strain enters into the logarithm phase, the precursor, such as phenylpropyl amino acid, benzamine, sodium benzoate, sodium acetate, and acetamide etc., can be added. Preferably, the concentration (versus culture medium) is 0.005%˜0.1%. The basic solution can be used to regulate the pH during the process of the fermentation.

The said fermentation can be performed by using the conventional or well-known apparatus under the conventional or well-known condition in the art. For example, the method of shake-flask can be used with the conventional or well-known rotate speed in the art; also, it can be performed in the conventional fermentation tank, for example, 7 L, 50 L fermentation tanks.

When 7 L or 50 L fermentation is used during the said fermentation, the carbon source can be added during the fermentation. It is not necessary to regulate the pH in the prophase of the fermentation, and the pH can be regulated to 6.0˜7.0 in the metaphase and anaphase. The method of inoculating the hyphae into the tank can be used. Relatively high airflow may be used before the plateau phase of growth. After the plateau phase, the airflow should be reduced.

To isolate the inventive novel compound of formula (I) from the culture medium, any conventional or well-known method for isolating secondary metabolic products from the culture medium in the art can be used. For example, the microorganism which comprises mainly of hyphae can be isolated from the fermenting solution by centrifuging or by filtering. The inventive compound of formula (I) can be extracted from the filtrate and the supernatant culture medium by one or more organic solutions (for example, alkyl halide, such as methylene chloride, methenyl chloride etc.). On the other aspect, the inventive compound of formula (I) mixed in the isolated hypha can be extracted from the hypha by one or more organic solutions (for example, acetone, ethyl acetate or methanol). The obtained inventive compound of formula (I) can be purified by the conventional or well-known method in the art, for example, by chromatography or crystallization.

The method of extracting and purifying the inventive novel compound of formula (I) from the said cell and culture medium comprises the following steps of: (1) isolating the fermented mycelia and the fermented supernatant from the culture medium obtained from the process of the said fermentation; (2) Extracting from the obtained fermented mycelia by using the first organic solution; extracting from the obtained fermented supernatant by using the second organic solution; drying by vaporizing the solution; (3) Purifying the product obtained from the second step by the method of chromatogram and crystallization to acquire the inventive novel compound of formula (I).

After the said fermentation is finished, the pretreatment can be performed at or below the room temperature, preferably below the room temperature, the pH of the fermenting solution is adjusted to 2˜9 by diluted acid or basic solution, with or without light, preferably without light. The said isolating process will be performed after the pretreatment. During the said step (1), the isolation can be performed by the conventional or well-known method, for example, centrifuge or filter the obtained fermentation solution.

In the said step (2), the obtained fermented mycelia were dried and crushed, and then extracted by using the organic solution. The said dryness can be performed by the conventional or well-known method in the art for example, dry it in air, heat it below 60° C. to dry, dry it in the freezing vacuum, the freezing vacuum is preferable, preferably performed without light. The said first organic solution which is used in extracting the inventive compound of formula (I) from the fermented mycelia can be the conventional or well-known solution in the art, for example, acetone, ethyl acetate, methanol, ethanol, methylene chloride and chloroform and the like, ethyl acetate is preferable. The said second organic solution used to extract the inventive compound of formula (I) from the fermented supernatant can be the conventional or well-known solution in the art, for example, methylene chloride and chloroform and the like. The said dryness can be performed by the conventional or well-known method in the art, for example, drying in air, concentrating by decompression.

In the said step (3), the purification by chromatogram can be performed by the conventional or well-known method in the art by means of silica gel or alumina column, and ethane/ethyl acetate, methanol/methylene chloride, ethanol/methylene chloride, acetone/methylene chloride and the like can be also used in the flowing phase. Also, more than one chromatogram column can be used.

The method of crystallization conventional or well known in the art. The process of the said crystallization is to: dissolve the obtained product in the solution such as methanol and the like, then add some water, set it in 4˜10° C. for 1˜12 hours, the crystal of the inventive novel compound of formula (I) is separated out, and then filtered. The process is repeated until the purification of the product is relatively high, then filter and dry the product in vacuum, finally the purified inventive compound of formula (I) is obtained.

Alternatively, the process of the said crystallization can also be as follows: dissolve the obtained product in the solution such as ethyl acetate and the like, then add the solvent such as petroleum ether or the like, decrease the temperate to 4˜10° C. to separate the crystals of the inventive compound, and then filter the product. The process is repeated until the desired high purity of the product obtained. The crystals are then filtered and dried under reduced pressure or in a vacuum as described above.

The results of determining the in vitro activity of the compound of formula (I) show that the compound has the bioactive properties of being anti-cancer, anti-fungi, anti-viral and the like.

Therefore, the present invention also provides an anti-cancer medicament comprising the inventive compound of formula (I).

The present invention also provides an anti-fungal medicament comprising the inventive compound of formula (I).

The present invention also provides an anti-viral medicament comprising the inventive compound of formula (I).

The present invention also provides methods for the manufacturing of the medicaments employed for anti-cancer, anti-fungal, and/or anti-viral uses.

Due to its unique stereo chemical structure, the inventive novel compound has unique bioactivities, which are significantly different from that of the previously described compound of formula (b). The well-known compound of formula (b) is considered as a biosynthetic inhibitor of β-1,3-glucan, and the well-known compound of formula (a) is considered as growth regulator of plants. The inventive novel compound has extremely strong bioactivity against various cancers such as stomach cancer, leukemia, and ovary cancer. Meanwhile, the compound also has high anti-fungal and anti-viral effect, which makes it useful in exploring a new generation of anti-cancer, anti-fungal, and anti-viral medicaments. The range of potential uses of the compound is broad.

The present invention is further explained by the following examples. However, the present invention is not limited to these examples in any manner. Those skilled in the art will understand that the equivalent substitutes to the specific substances described herein, or the corresponding improvements are considered to be within the scope of the invention, and are covered by the following claims.

In this description, the terms “strain” and “microorganism” refer to the fungal strain of the present invention unless otherwise specified. The following examples will show how the culture and extraction conditions were varied to optimize production of the compound of formula (I), how the compound was purified and how its bioactivities were determined.

The methods used in the present invention are well-known in mycology, organic chemistry and biomedical sciences with the respective parameters varied and optimized for the present invention.

### EXAMPLES

### Example 1

### Isolation of the Microorganism Producing Taxol from the Taxus Yunnanensis Samples

The inventors collected 310 samples from the branches, barks, leaves and roots of seven Taxus yunnanensis trees that are over 300-year-old growing in the conifer and broad-leaf mixed forest in 2500˜3000 meter zone above sea level of LaoJun Mountain in Lijiang Region of Yunna Province, China. Under sterile conditions, the pieces were sterilized with 60% ethanol, washed by sterile water, dried by aspirating the extra water and then the tissue was torn into many thin slices layer by layer. The samples were cut into little parts, arranged in the order of from the inter tissue layers to the outer layers, and then the samples were inoculated into the liquid agar medium, cultured at 25° C. for 7˜15 days as described above. Alternatively the samples were cut into small blocks approximately of size 1 cm×1 cm, further sterilize by using 50˜90% ethanol for 5 min, homogenized by homogenizer, and the homogenized samples were spread onto the PDA culture medium.

The samples were incubated in dish at 25° C. for 4˜5 days, and the grown hyphae were transferred onto the sloped surface of the culture medium in culture tubes, incubated at 20° C.˜35° C. for four days, and 1 cm² of mycelia was removed into another medium (for a 250 ml flask, the total volume of the culture medium is 100 ml; the ingredients of the medium are: 2% glucose, 0.5% powdered soybean, 0.2% powdered peanut, 0.01% MgSO₄, and 0.05% KH₂PO₄), cultured by shaking at 300 rpm at 25° C. for 16 days. The wet mycelia were obtained after being filtered by two-layers of gauze. The wet mycelia were dried by heating at 60° C., and then immersed in ethyl acetate (100 ml chloroform was added on the basis of 20 grams dried mycelia) for 12 hours. The mycelia were concentrated in a vacuum until the dried extract was obtained. The dried extract was dissolved in 1 ml methanol solution and 5F1 of the solution was dropped onto the silica gel matrix of TLC plates. Analysis was performed under three different thin-layer chromatographic solvent systems (ethyl acetate:isopropanol=95:5 v/v, chloroform:methanol=7:3 v/v, chloroform:acetonitrile=7:3 v/v). After that, the plates were dried at 60° C. for 30 minutes, they were then stained with 5% sulphuric acid ethanol solution. Spots on the TLC plates whose moving speed is the same as those of the compound of formula (I) and of taxol were picked up by scraping and further identification of the strain from which these compounds were obtained were performed. The final identification was performed to select the endogenous fungi which can produce the compound of formula (I) and taxol.

### Example 2

### Purification and Identification of the Novel Fungal Strain Producing the Compound of Formula (I)

The ST-026 strain was obtained after several kinds of fungal strains producing taxol was obtained from example 1 were isolated and purified for three generations (20˜35° C., for 4˜10 days) on the PDA culture dish. The ST-026 strain grows very fast and produces plenty of sporules in the PDA medium. Usually the sporules are black or olive green. The peduncle of the conidiophore grows up directly from the agar, and the chain of the conidiophore grows up from the side branch of the hypha. The chain of the sporule comprises 50˜70 sporules. The initial sporule grows up directly from the peduncle of the sporule, or from the surfaces of 1 or 2 sporules. The sporule is claviform, olivary or elliptic with the size of around 22˜33(25)×9˜13(11) μm, having 4˜5 horizontal compartments and 2˜3 vertical or sidelong compartments. The initially formed young sporule is narrowed olivary, having dense, tiny wart, which makes the horizontal compartments of the sporule unclear under the microscope (100×). Usually the hypha is colorless or olive green, having compartments, straight. The length of the hypha cell is 5 times longer than the width. Initially, the branch of the hypha is horizontal with a long, sharp top. According to the above characters and ALTERNARIA ALTERNATA founded by KEISSLER in 1912, the strain is identified as Alternaria alternata var. monosporus.

As an initial strain, the strain was induced by ultraviolet radiation. A mutant strain, UV-012 was obtained after 12 generations. By using the UV-012 as an initial strain to continue the mutant selection, the monosporus strain, code number ST-026, which can stably produce the inventive novel compound and taxol was obtained. The above mutant strain was stable after five generations of being cultured. According to conventional plant nomenclature, the strain was named as Alternaria alternata var. monosporus, the code number was ST-026-R. The strain is deposited in China General Microbiological Culture Collection Center (CGMCC) at No. 13 Zhongguancun Yi Tiao, Beijing; Deposit No.: CGMCC 0899.

### Example 3

### Seed Culture

The lyophilized strain of CGMCC No. 0899 obtained from the example 2 was inoculated into a 250 ml-flask containing 100 ml seed culture medium (Table 1) and culturing at 20˜30° C. for 24˜48 hours with the shaking speed of 100˜300 rpm.

TABLE 1
Seed culture medium

| Component | % |
|---|---|
| Glucose | 2 |
| Powdered soybean | 0.5 |
| Powdered peanut | 0.2 |
| MgSO₄ | 0.01 |
| NaH₂PO₄ | 0.05 |
| Deionized water | 100 ml |
| Adjust pH to | 7.0~7.2 |

### Example 4

### Shake-Flask Fermentation

The strain obtained from the example 2 was cultured on slant PDA seed culture medium. The 7-day-old mycelia with approximately 2×10⁶ spores/100 ml of culture medium were directly inoculated into the culture medium (components: 50 g/L of sucrose, 2.5 g/L of powdered soybean, 0.5 g/L of yeast powder, 0.3 g/L of magnesium sulfate, 0.35 g/L of monopotassium phosphate, 0.56 g/L of dipotassium phosphate, 10 mg/L of Vitamin B₁, 2 mg/L of ferric trichloride, 5 mg/L of manganese sulfate, 2.5 mg/L of zinc sulfate, 0.7 mg/L of potassium iodide, 1.4 mg/L of boric acid, and pH6.8). One or more inducers such as arachidonic acid (0.01%), cerous ammonium nitrate (0.02%), potassium permanganate (0.02%) were added during inoculation.

A total volume of 100 ml in a 250 ml-flask was incubated at 25° C. with the rotation speed of 150 rpm. Four days after inoculating, 0.005% of benzamine, 0.01% of phenylpropyl amino acid and 0.04% of sodium acetate were added. The duration of the fermentation was nine days. The products were purified according to the method described in example 24. Results: The concentration of the mycelia is 0.50 g/L, the yield of the product represented by formula (I) is 780 mg/L.

### Examples 5-8

### Shake-Flask Fermentation

The shake-flask fermentation was performed according to the method described in Example 4 except that the culture media consisted respectively of:

Example 5: 50 g/L of sucrose, 2.5 g/L of powdered soybean, 0.5 g/L of yeast extract, 0.06 g/L of magnesium sulfate, 1.2 g/L of calcium carbonate, 10 mg/L of Vitamin B1, 2 mg/L of ferric trichloride, 5 mg/L of manganese sulfate, 2.5 mg/L of zinc sulfate, 0.7 mg/L of potassium iodide, and 1.4 mg/L of boric acid, and pH 6.8. Results: The concentration of mycelia is 45 g/L, the yield of the product represented by formula (I) is 700 mg/L.

Example 6: 10 g/L of glucose, 30 g/L of sucrose, 10 g/L of powdered soybean, 0.5 g/L of yeast extract, 0.1 g/L of magnesium sulfate, 10 mg/L of Vitamin B₁, 0.56 g/L of monopotassium phosphate, 0.35 g/L of dipotassium phosphate, 2 mg/L of ferric trichloride, 5 mg/L of manganese sulfate, 2.5 mg/L of zinc sulfate, 0.7 mg/L of potassium iodide, and 1.4 mg/L of boric acid, and pH 6.8.

Results: The concentration of mycelia is 48 g/L, the yield of the product represented by formula (I) is 750 mg/L.

Example 7: 50 g/L of sucrose, 5 g/L of powdered soybean, 0.5 g/L of yeast extract, 1 g/L of magnesium sulfate, 0.6 g/L of monopotassium phosphate, 5 g/L of potassium sodium tartrate, 10 mg/L of Vitamin B₁, 2 mg/L of ferric trichloride, 5 mg/L of manganese sulfate, 2.5 mg/L of zinc sulfate, 0.7 mg/L of potassium iodide, and 1.4 mg/L of boric acid, and pH 6.8.

Results: The concentration of mycelia is 50 g/L, the yield of the product represented by formula (I) is 760 mg/L.

Example 8: 5 g/L of glucose, 20 g/L of sucrose, 10 g/L of dissolvable starch, 3 g/L of powdered soybean, 0.5 g/L of yeast extract, 0.1 g/L of magnesium sulfate, 10 mg/L of Vitamin B₁, 0.56 g/L of monopotassium phosphate, 0.35 g/L of dipotassium phosphate, 2 mg/L of ferric trichloride, 5 mg/L of manganese sulfate, 2.5 mg/L of zinc sulfate, 0.7 mg/L of potassium iodide, and 1.4 mg/L of boric acid, and pH 6.8.

Results: The concentration of mycelia is 47 g/L, the yield of the product represented as formula (I) is 760 mg/L.

The amount of inducer added while inoculating is indicated respectively:

Example 5: 0.005% of arachidonic acid, 0.06% of cerous ammonium nitrate and 0.01% of potassium permanganate;

Example 6: 0.01% of methyl jasmine, 0.006% of cerous ammonium nitrate and 0.02% of potassium permanganate;

Example 7: 0.05% of arachidonic acid, 0.005% of ammonium citrate, and 0.008% of potassium permanganate;

Example 8: 0.05% of ammonium citrate, and 0.02% of cerous ammonium nitrate.

The amount of precursor added in the fourth day after inoculating are respectively, For Example 5: 0.008% of benzamine, 0.02% phenylpropyl amino acid, and 0.02% of sodium acetate;

For Example 6: 0.007% of phenylpropyl amino acid, 0.05% of sodium benzoate, and 0.01% of acetamide;

For Example 7: 0.02% of benzamine, 0.03% of sodium acetate, and 0.005% of acetamide;

For Example 8: 0.002% of phenylpropyl amino acid, 0.01% of sodium acetate, and 0.09% of acetamide.

### Example 9

### 7 L Tank Fermentation

A primary seed culture was obtained after 24-hour of culturing according to the method described in Example 3 except that the culture temperature was 25° C., and the rotation speed was 150 rpm. According to the method of tank fermentation, 10% of the obtained hyphae were inoculated into the 7 L-fermentation tank with the culture medium including 10 g/L of glucose, 20 g/L of sucrose, 2.5 g/L of powdered soybean, 0.5 g/L of yeast extract, 0.3 g/L of magnesium sulfate, 0.35 g/L of monopotassium phosphate, 0.56 g/L of dipotassium phosphate, 10 mg/L of Vitamin B₁, 2 mg/L of ferric trichloride, 5 mg/L of manganese sulfate, 2.5 mg/L of zinc sulfate, 0.7 mg/L of potassium iodide, 1.4 mg/L of boric acid and pH is 6.8. The inducers added at the same time were: 0.01% of arachidonic acid, 0.02% of cerous ammonium nitrate, and 0.02% of potassium permanganate. It was not necessary to adjust the pH during the prophase of the fermentation, however, the pH was adjusted to 6.0˜7.0 with sodium bicarbonate solution 24 hours after the plateau phase of growth. Because abundant air flow was required before the plateau phase of growth, it was beneficial for the synthesis of the new compound of formula (I) that the concentration of the oxygen be raised to above 30% before the plateau phase, and then reduced to below 20% after the plateau phase. Thirty-six hours after inoculating, 1% of sucrose was added to the circulating medium for 24 hours. On the sixth day of the inoculation, 2% of sucrose and 2% of maltose were added once. The precursors added at the 96th hour of the fermentation were: 0.005% of benzamine, 0.01% of phenylpropyl amino acid, 0.04% sodium acetate one-off. The duration of the fermentation was nine days. Results: The yield of the new compound of formula (I) was 805 mg/L.

### Example 10

### 7 L Tank Fermentation

The same seed culture obtained from the example 3 were cultured in conventional sporule-culture medium (rice, glucose, agar) at 23˜29° C. for 5˜9 days, the concentration was 2×10⁷ sporules/L of medium. Using the method of directly adding the sporules into the tank, the obtained sporules were inoculated into the medium of 7 L-tank fermentation (including 10 g/L of glucose, 20 g/L of sucrose, 2.5 g/L of powder of the soybean, 0.5 g/L of yeast extract, 0.3 g/L of magnesium sulfate, 0.35 g/L of monopotassium phosphate, 0.56 g/L of dipotassium phosphate, 10 mg/L of Vitamin B₁, 2 mg/L of ferric trichloride, 5 mg/L of manganese sulfate, 2.5 mg/L of zinc sulfate, 0.7 mg/L of potassium iodide, and 1.4 mg/L of boric acid, pH 6.8), with 0.01% of acetamide added. The pH was adjusted to 6.0˜7.0 with 2N of sodium hydroxide solution and 2N of hydrochloride acid solution during the process of the fermentation. The oxygen concentration was controlled to above 30% by mediating the air flow until the second half of the growth plateau phase. Additionally the concentration of the oxygen was reduced to below 20% after the second half of the plateau phase. Thirty-six hours after inoculating, 1% of sucrose was added to the circulating medium for 24 hours; On the sixth day of the inoculation, 1% of culture medium was added to the circulation for 24 hours, and then 0.1% of acetamide as a precursor was added at the second half of the plateau phase. The duration of the fermentation was nine days. Results: The yield of the new compound of formula (I) was 760 mg/L.

### Example 11

### 7 L Tank Fermentation

The same method was used as described in the example 10, except that the medium of the fermentation included: 50 g/L of sucrose, 2.5 g/L of powdered soybean, 0.5 g/L of yeast extract, 0.06 g/L of magnesium sulfate, 3.0 g/L of calcium carbonate, 10 mg/L of Vitamin B₁, 2 mg/L of ferric trichloride, 5 mg/L of manganese sulfate, 2.5 mg/L of zinc sulfate, 0.7 mg/L of potassium iodide, and 1.4 mg/L of boric acid, its pH was 6.8. The inducers added were: 0.09% of acetamide, 0.01% of potassium permanganate, 0.01% of cerous ammonium nitrate and 0.005% of arachidonic acid. The pH was adjusted to 6.0˜7.0 with 2N of sodium hydroxide solution and 2N of hydrochloride acid solution during the process of the fermentation. The oxygen concentration was controlled to above 30% by mediating the air flow until the second half of the plateau phase, and the concentration of the oxygen was controlled to below 20% after the second half of the plateau phase. 0.1% of acetamide as a precursor was added one-off at the second half of the plateau phase. The duration of the fermentation was nine days. Results: The yield of the new compound of formula (I) was 755 mg/L.

### Example 12

### 7 L Tank Fermentation

The same method was used for obtaining primary seed culture with the method described in example 3, the culture medium contained 20 g/L of glucose, 5 g/L of powdered soybean, 2 g/L of powdered peanut, 0.1 g/L of magnesium sulfate and 0.5 g/L of sodium dihydrogen phosphate, its pH is 6.8. The seeds were cultured at 25° C. with the rotation speed of 250 rpm for 16 hours.

Fifteen percent of the obtained seeds were inoculated into a 7 L fermentation tank by using the conventional method for inserting the hyphae into the tank directly. The medium contained 20 g/L of sucrose, 2.5 g/L of powdered soybean, 0.5 g/L of yeast extract, 0.06 g/L of magnesium sulfate, 3.0 g/L of calcium carbonate, 10 mg/L of Vitamin B₁, 2 mg/L of ferric trichloride, 5 mg/L of manganese sulfate, 2.5 mg/L of zinc sulfate, 0.7 mg/L of potassium iodide and 1.4 mg/L of boric acid, the pH thereof was 6.8. The inducers added were: 0.01% of acetamide, 0.002% of potassium permanganate, 0.005% of cerous ammonium nitrate and 0.005% of arachidonic acid. The pH was adjusted to 6.0˜7.0 with 2N sodium hydroxide solution or 2N hydrochloride acid solution during the process of the fermentation. The oxygen concentration was controlled to above 30% by mediating the air flow until the second half of the plateau phase, and the concentration of the oxygen was controlled to below 20% after the second half of the plateau phase. In 36 hours after inoculating, 2% of sucrose was added to the circulating medium for 24 hours; at the 96th hour, 0.1% of acetamide, a precursor, was added one-off, and 1% of the medium was also added to the circulation for 12 hours. The duration of the fermentation was nine days. Results: The yield of the new compound of formula (I) was 758 mg/L.

### Example 13

### 7 L Tank Fermentation

The same method was applied as described in the example 12, except that the inducers added were: 0.02% of potassium permanganate, 0.05% of cerous ammonium nitrate and 0.005% of arachidonic acid. 2% of sucrose was added to the circulating medium for 24 hours 36 hours after the start of the fermentation, at 96 hours after the start of fermentation, precursors including 0.01% phenylpropyl amino acid, 0.04% of sodium benzoate, 0.01% of acetate were added. At the same time 1% of the medium was also added to the circulation. The duration of the fermentation was nine days. Results: The yield of the new compound of formula (I) was 765 mg/L.

### Example 14

### 50 L Tank Fermentation

The seeds were cultured by using the same method as described in example 3. The primary seeds were inoculated into the medium comprising 10 g/L of glucose, 30 g/L of sucrose, 2.5 g/L of powdered soybean, 0.5 g/L of yeast extract, 0.3 g/L of magnesium sulfate, 0.56 g/L of dipotassium hydrogen phosphate anhydrous 0.35 g/L of potassium dihydrogen phosphate, 10 mg/L of Vitamin B₁, 2 mg/L of ferric trichloride, 5 mg/L of manganese sulfate, 2.5 mg/L of zinc sulfate, 0.7 mg/L of potassium iodide and 1.4 mg/L of boric acid, the pH thereof was 6.8. The added inducers were: 0.01% arachidonic acid, 0.05% cerous ammonium nitrate and 0.02% potassium permanganate. At 36 hours after inoculating, 1% of sucrose and 0.2% of yeast extract were added for 24 hours to the circulating medium. On the 6^{th} day 2% sucrose and 2% maltose one-off were added. On the 96 hour of fermentation, precursors comprising 0.005% of benzamine, 0.01% of phenylpropyl amino acid, and 0.04% of sodium acetate was added one-off. After adding the precursors, the pH thereof was adjusted to 6.0˜7.0 by using sodium hydroxide solution. The duration of the fermentation was 9 days. From the beginning of the fermentation to the second half of the plateau phase, plenty of air flow was required, It was beneficial for synthesizing the new compound of formula (I) that the oxygen concentration be controlled to above 30% before and during the first half of the plateau phase and the concentration of the oxygen reduced to below 20% after the second half of the plateau phase. Results: The yield of the novel compound of formula (I) was 780 mg/L.

### Example 15

### 50 L tank fermentation

The same method was used as described in example 14 for 50 L tank fermentation, except that the medium contained 20 g/L of glucose, 5 g/L of powdered soybean, 2 g/L of powdered peanut, 0.1 g/L of magnesium sulfate and 0.5 g/L of sodium dihydrogen phosphate, pH was 6.8. The seeds were cultured at 25° C. with the rotation speed of 250 rpm for 16 hours.

Fifteen percent of the obtained hyphae were inoculated into the 50 L fermentation tank by using the conventional method for inserting the hypha into the tank. The medium contained 10 g/L of glucose, 20 g/L of sucrose, 2.5 g/L of powdered soybean, 0.5 g/L of yeast extract, 0.06 g/L of magnesium sulfate, 3.0 g/L of calcium carbonate, 10 mg/L of Vitamin B₁, 2 mg/L of ferric trichloride, 5 mg/L of manganese sulfate, 2.5 mg/L of zinc sulfate, 0.7 mg/L of potassium iodide and 1.4 mg/L of boric acid, pH was 6.8. The inducers added were 0.01% of acetamide, 0.02% of potassium permanganate, 0.05% of cerous ammonium nitrate and 0.005% of arachidonic acid. The pH thereof was adjusted to 6.0˜7.0 by using sodium hydroxide and hydrochloric acid during fermentation. The oxygen concentration was controlled to above 30% by mediating the air flow until the second half of the plateau phase, and the concentration of the oxygen was also controlled to below 20% after the second half of the plateau phase. In 36 hours after inoculating, 2% of sucrose and 0.1% yeast extract were added to the circulating medium for 24 hours. At the 96 hours, 0.1% of acetamide, a precursor was added one-off, and then 1% of the medium was added to the circulation for 12 hours simultaneously. The duration of the fermentation was nine days. Results: The yield of the new compound of formula (I) was 740 mg/L.

### Example 16

### 50 L Tank Fermentation

The same method was used as described in example 14 for 50 L tank fermentation, except that the medium contained 10 g/L of glucose, 30 g/L of sucrose, 5 g/L of powdered soybean, 2 g/L of powdered peanut, 0.1 g/L of magnesium sulfate, 0.5 g/L of sodium dihydrogen phosphate, pH was 6.8. The seeds were cultured at 25° C. with the rotation speed of 250 rpm for 16 hours.

Fifteen percent of the obtained hyphae were inoculated into the 50 L fermentation tank by using the conventional method for inserting the hyphae into the tank. The medium contained 10 g/L of glucose, 40 g/L of sucrose, 5 g/L of powdered soybean, 0.5 g/L of yeast extract, 0.06 g/L of magnesium sulfate, 3.0 g/L of calcium carbonate, 10 mg/L of Vitamin B1, 2 mg/L of ferric trichloride, 5 mg/L of manganese sulfate, 2.5 mg/L of zinc sulfate, 0.7 mg/L of potassium iodide and 1.4 mg/L boric acid, pH was 6.8. The inducers added were 0.01% of acetamide, 0.02% of potassium permanganate, 0.05% of cerous ammonium nitrate and 0.005% of arachidonic acid. The pH was adjusted to 6.0˜7.0 by using sodium hydroxide and hydrochloric acid during the fermentation. The oxygen concentration was controlled to above 30% by mediating the air flow until the second half of the plateau phase, and the concentration of the oxygen was also controlled to below 20% after the second half of the plateau phase. At 96 hours, 0.1% of acetamide, a precursor was added one-off, and 1% of the medium was added to the circulation for 12 hours simultaneously. The duration of the fermentation was nine days. Results: The yield of the new compound of formula (I) was 738 mg/L.

### Example 17

### 50 L Tank Fermentation

The same method was used as described in example 14 for 50 L tank fermentation, except that the medium contained 10 g/L of glucose, 30 g/L of sucrose, 5 g/L of powdered soybean, 0.1 g/L of magnesium sulfate and 0.5 g/L of sodium dihydrogen phosphate, pH was 6.8. Or other conventional medium could also be used. The seeds were cultured at 25° C. with the rotation speed of 250 rpm for 16 hours.

Fifteen percent of the obtained hyphae were inoculated into the 50 L fermentation tank by using the conventional method for inserting the hypha into the tank. The medium contained 50 g/L of sucrose, 2.5 g/L of powdered soybean, 0.5 g/L of yeast extract, 0.06 g/L of magnesium sulfate, 3.0 g/L of calcium carbonate, 10 mg/L of Vitamin B₁, 2 mg/L of ferric trichloride, 5 mg/L of manganese sulfate, 2.5 mg/L of zinc sulfate, 0.7 mg/L of potassium iodide, and 1.4 mg/L of boric acid, pH was 6.8. The inducers added were 0.03% of acetamide. its the pH was adjusted to 6.0˜7.0 by using sodium hydroxide and hydrochloric acid during the fermentation. The oxygen concentration was controlled to above 30% by mediating the air flow until the second half of the plateau phase, and the concentration of the oxygen was then controlled to below 20% after the second half of the plateau phase. At 96 hours 0.1% of acetamide as a precursor was added one-off, and 1% of the medium was also added to the circulation for 12 hours simultaneously. The duration of the fermentation was nine days. Results: The yield of the new compound of formula (I) was 725 mg/L.

### Example 18

### 50 L Tank Fermentation

The same method was used as described in example 14 for 50 L tank fermentation, except that the medium contained 20 g/L of glucose, 5 g/L of powdered soybean, 2 g/L of powdered peanut, 0.1 g/L of magnesium sulfate and 0.5 g/L of sodium dihydrogen phosphate, pH was 6.8. The seeds were cultured at 25° C. with the rotation speed of 250 rpm for 16 hours.

Fifteen percent of the obtained hyphae were inoculated into the 50 L fermentation tank by using the conventional method for inserting the hypha into the tank. The medium contained 10 g/L of glucose, 20 g/L of sucrose, 2.5 g/L of powdered soybean, 0.5 g/L of yeast extract, 0.06 g/L of magnesium sulfate, 0.56 g/L of dipotassium hydrogen phosphate anhydrous 0.35 g/L of potassium dihydrogen phosphate, 10 mg/L of Vitamin B₁, 2 mg/L of ferric trichloride, 5 mg/L of manganese sulfate, 2.5 mg/L of zinc sulfate, 0.7 mg/L of potassium iodide and 1.4 mg/L of boric acid, pH was 6.8. The pH was adjusted to 6.0˜7.0 by using sodium hydroxide and hydrochloric acid during the fermentation. The oxygen concentration was controlled to above 30% by mediating the air flow until the second half of the plateau phase, and the concentration of the oxygen was also controlled to below 20% after the second half of the plateau phase. In 36 hours after inoculating, 2% of sucrose and 0.1% yeast extract were added to the circulating medium for 24 hours. At 96 hours the precursor including 0.01% of phenypropyl amino acid, 0.04% of sodium benzoate, 0.05% sodium acetate was added one-off, and simultaneously, 1% of the medium was also added to the circulation for 12 hours. The duration of the fermentation was nine days. Results: The yield of the new compound of formula (I) was 730 mg/L.

### Examples 19˜23

### Isolation and Purification of the Product

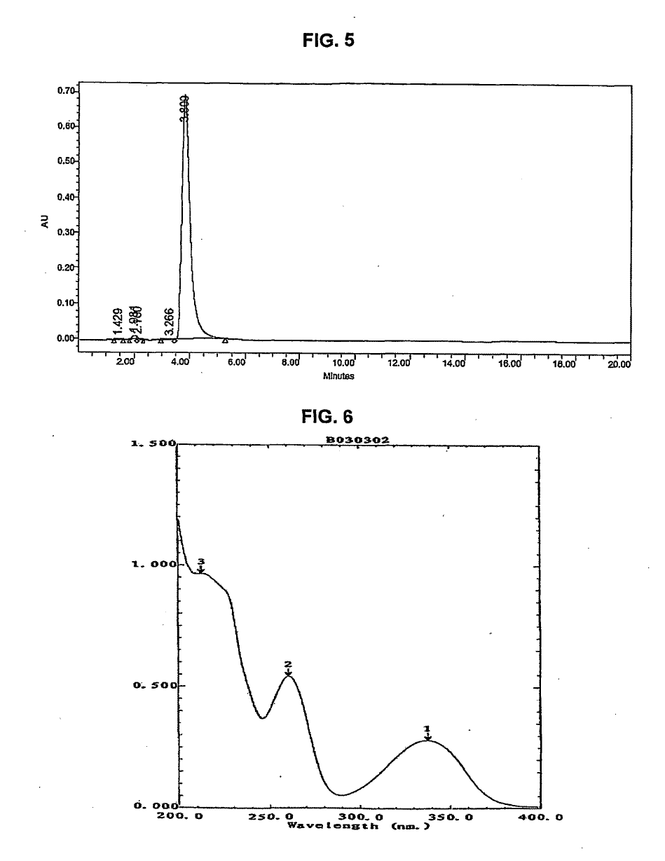

The fermented solutions obtained from the examples 9˜13 were filtered, followed by collecting the filtered fermented solution and the supernatant. From the fermented solutions of the respective examples, 151 g/7 L, 146 g/7 L, 145 g/7 L, 143 g/7 L, 142 g/7 L of the dried mycelia were obtained by drying in air, freeze drying (lyophilization), by heating to 60° C., by heating to 80° C. and by heating to 100° C., respectively. The dried mycelia were crushed and added at the following solvents (at a mycelia:solution ratio of 1:20) ethyl acetate, acetone, ethanol, methanol, ethyl acetate, respectively. The mycelia were extracted for 24, 26, 30, 30, 25 hours respectively. The respective extracts were obtained by evaporation of the solvents under reduced pressure. A representative TLC result of the extract obtained from an example is shown in FIG. 4. A representative high performance liquid chromatography (HPLC) spectrum graph is shown in FIG. 5. From the figures, it is easily to understand that the sample contains large amount of the compound of formula (I). The dots or peaks indicated by the arrows in the FIGS. 4 and 5 indicate the characteristic dots or peaks of the novel compound of formula (I).

An equal volume of methylene chloride or chloroform was added into the supernatant obtained from the above filtered solution, followed while extracting by shaking for 1˜2 hours, and then centrifuging at 8000 rpm for 10˜30 min. The sample was then placed on a table for 10 min and the organic solution layer was thus obtained. The extract was obtained by evaporating the solvent under reduced pressure.

The materials obtained above was dissolved with the mobile phase, and then the solution was applied to a chromatographic column of silica gel or alumina (200˜400 mesh, height and diameter of the column is 5×40 cm), the mobile phase was: 1%˜10% ethanol/CH₂Cl₂ or methanol/CH₂Cl₂, 1%˜30% acetone/CH₂Cl₂; gradient elution was performed under medium pressure with a rate of 40 ml/min, collecting the purified product, and evaporating under reduced pressure to concentrate and hence obtain the product. The novel compound of formula (I) was obtained with a purity of 92%.

The compound of formula (I) with the purity of 92% was then dissolved in methanol solution, and distilled water was added slowly with heating to 40˜60° C. The addition of distilled water was stopped with the crystals of the compound was separated out. The solution was cooled to 4˜10° C., and was filtered. The solution was further dried under reduced pressure and dried to obtain the novel compound of formula (I) with the purity of 98%. The above procedure was repeated two to three times, followed by filtering, evaporation under reduced pressure and drying to finally obtain the novel compound of formula (I) with the purity of 99.5%.

### Example 24-25

### Isolation and Purification of the Product

The fermented solutions obtained from Examples 9˜13 were centrifuged and the filtered fermented solution and the supernatant were collected. The obtained mycelia was then freezed dried. Thereafter, the dried mycelia was crushed and ethyl acetate was added with shaking for 12 hours. This extract was then filtered and evaporating the solution.

Equal volumes of methylene chloride or chloroform was added into the supernatant obtained from the above filtered solution. After extracting by shaking for 1˜2 hours, and the organic solution layer was obtained by centrifuging. This was then reduced under pressure and evaporated to obtain an extract.

The extracts described above were dissolved in solvents of the mobile phase, and then applied to the chromatographic column containing silica gel or alumina (200˜400 mesh, height and diameter of the column is 5×40 cm), the mobile phase was: 1%˜10% acetone/CH₂Cl₂; gradient elution was performed under medium pressure with a rate of 20-60 ml/min. After evaporation under reduced pressure and drying, the novel compound of formula (I) was obtained with the purity of 92%.

The compound of formula (I) with the purity of 92% was dissolved in methanol solution, and 1.5˜2 times volume of petroleum ether was added slowly. As the crystal of the compound disappeared, the solution was cooled to 4˜10° C., and was filtered. The above procedure was repeated two to four times. After filtering, evaporation under reduced pressure and drying, the novel compound of formula (I) with the purity of 99.5% was finally obtained.

### Example 26

### Isolation and Purification of the Product

The same method as described in Example 24 was used to isolate and purify the products obtained from the Examples 4 to 8, and 16 to 18 to produce a substantially pure compound of (I).

### Example 27

### Isolation and Purification of the Product

The same methods as described in examples 19 to 26 were used to isolate and purify the products obtained from the Examples 10 to 17, except that before isolating the products obtained from the Example 10 to 17, 1 mol/L of hydrochloric acid or 1 mol/L sodium hydroxide was added slowly, with constant stirring, and pH was adjusted to 6.8, 6.5, 5.5, 4.5, 3.0, 7.5, 8.6, 6.0 respectively. This step was performed in darkness. A substantially pure compound of formula (I) was obtained.

### Example 28

### Identification of the Product

The products obtained from the examples 19 to 27 were identified and characterized by using ultra-violet (UV), infra-red (IR), mass spectroscopy (MS), nuclear magnetic resonance (NMR) spectroscopy and X-ray diffraction. The results are shown in FIGS. 6˜9. It can be concluded that the compound having formula (I) produced by using the methods of the present invention has the structure of the compound of formula (I). These data and characteristic spectra indicate how the compound of formula (I) may be identified from other compounds.

### Example 29

### The Anticancer Effects of the Compound of (I) In Vitro

The study of the anticancer effect of the compound of formula (I) was as follows.

1. Method: 3-[4,5-dimethyl(thiazol-2-yl)-3,5-diphery] tetradium bromide (MTT) assay.
2. Culture medium: RPMI-1640 medium containing 2% calf serum
3. Apparatus: (1) CO₂ incubator, Sherton USA; (2) 318MC Enzyme analysis machine, SANCO Equipments Company Ltd.
4. Conditions of the culture: Temp. 37° C.; Concentration of CO₂: 4.7%, culture duration: 24˜48 hours.

The primary selection results are shown in Table 2 and FIG. 11. FIG. 11 displays the percentage of cells killed by compound (I) for different cells.

The results show that the compound produced by the method of the present invention has very high activity for L1210 (mouse leukemia cells), B16 (mouse melanocytoma cells), A2780 (human ovary cancer cells) and MGC (human stomach cancer cells) The activities are 97.00%, 72.53%, 90.91%, 85.52%, respectively. It has no effect on Eca-109 (esophageal cancer cells). This indicates that the inventive novel compound has cell-selectivity in killing cancer cells, without toxicity. The compound can be developed an anticancer drug.

TABLE 2
The percentage of killing by using the compound
of formula (I) in different cells

| | | L1210 | Eca-109 | B16 | A2780 | MGC |
|---|---|---|---|---|---|---|
| Methanol | | 4.28% | 0% | 0% | 4.00% | 14.9% |
| 5 | ug/ml | 97.00% | 0.18% | 72.53% | 90.91% | 85.52% |
| 2.5 | ug/ml | 94.18% | 0% | 51.75% | 73.08% | 64.37% |
| 1.25 | ug/ml | 17.02% | 0% | 0% | 46.5% | 42.32% |
| 625 | ng/ml | 0% | 0% | 6.37% | 35.31% | 40.98% |
| 312.5 | ng/ml | 0% | 0% | 1.06% | 0% | 0% |

Note:
The wavelength used was 546 nm

### Example 30

### The Curative Effect of the Compound of Formula (I) in the Form of Lyophilized Powder for Injection in the Nude Mouse Transplanted with Human Stomach Cancer Cells MGC803

Aim: To observe the growth inhibitory effect and the intensity of the effect of the compound of formula (I) in nude mouse transplanted with human stomach cancer cells MGC803.

Material: The lyophilized injection powder of the compound of formula (I), 15 mg/vial, diluted to the needed concentration by 0.9% saline before use.

Dosages: the dosages of the compound of formula (I) are 15, 10 and 5 mg/kg.

Animals: BALB/cA nude mice, female, 40˜45-days old, body weight is 18±2 g. The number of the animals in per group: negative control: 12; group with drug: 6.

Transplanted tumor: Human stomach cancer MGC-803 cells were transplanted into the nude mice as follows. Approximately half a million (5×10⁶) of the MGC-803 cells was injected subcutaneously (s.c.) into the mice. The tumors were used after breeding the mice for three generations.

Methods: Tumors in peak growth were cut into the sizes of around 1.5 mm³. Under aseptic conditions the tumours were injected subcutaneously to the right axilla of the mouse. The size of the transplanted tumor is then measure and the animals randomly divided into several groups when the size of the tumor reaches 100-200 mm³. The drug is administered by intravenous (i.v.) injection on the 1st and 6th days. Saline is administered as control. The size of the tumor is examined every two weeks together with the weight of the mouse. The tumor volume(TV) is calculated as:

TV=1/2×a×b²

Where, a and b represent length and width, respectively. The relative tumor volume (RTV) is calculated as: RTV=Vt/V0, where V0 is the volume of the tumor before administering the compound, Vt is the volume of the tumor after administering the compound. The index for evaluating the activity of anticancer is the relative growth rate of tumor (T/C), which is calculated as:

T/C (%)=(T_{RTV}/C_{RTV})×100

wherein, T_{RTV} is the administering group; C_{RTV} is the control group.

The standard for evaluating the treatment effect: T/C (%)>60% means no effect, T/C<=60% and p<0.05 means having a significant effect.

### Results and Conclusions:

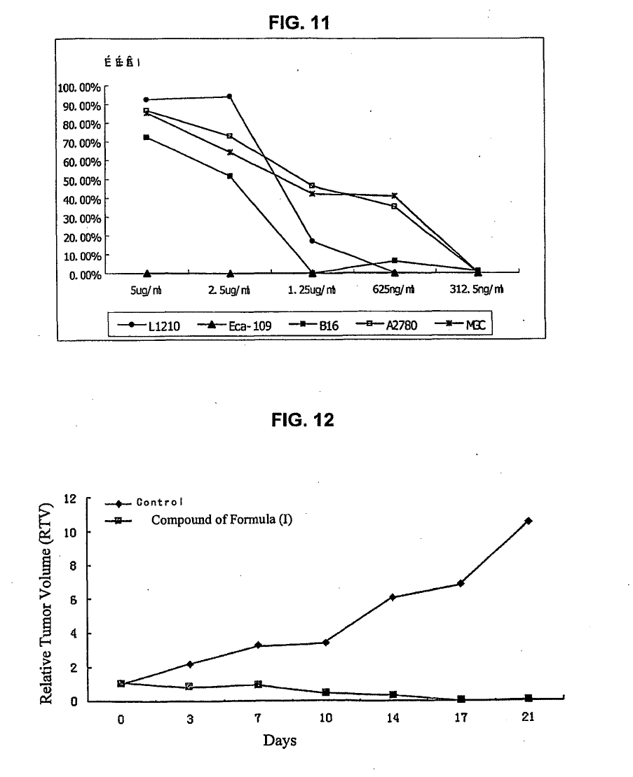

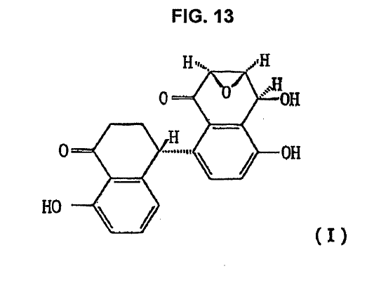

The experimental treatment effect of the compound of formula (I) in the form of lyophilized powder for injection on nude mice transplanted with human stomach cancer cells MGC-803 is shown in Table 3 and FIG. 12. The results indicate that administering of the lyophilized powder of the compound of formula (I) at the dose of 15 mg/kg on day 1 and day 6 by i.v. administration has significant growth inhibitory effect on nude mice transplanted with human stomach cancer cell MGC-803. The value of T/C is 0.18%. Three weeks after the treatment, the tumor in one mouse disappeared completely.

TABLE 3
The experimental treatment effect of lypholized powder of the compound of formula
(I) on the nude mice transplanted with human stomach cancer cells MGC-803

| | | | No. of animals | | weight(g) | | TV(mm³) | | | T/C |
|---|---|---|---|---|---|---|---|---|---|---|
| Group | Dose | Admin. | Initial | final | Initial | final | d 0 | d 21 | RTV | (%) |
| Negative | 0.2 ml/animal | i.v. | 12 | 12 | 19.0 | 19.8 | 107 ± 48 | 923 ± 475 | 10.5 ± 8.0 | |
| Control | | | | | | | | | | |
| Compound | 15 mg/kg, d 1, d 6 | i.v. | 6 | 6 | 18.3 | 17.2 | 102 ± 22 | 2.2 ± 0.04 | 0.02 ± 4.3 | 0.18 |
| (I) | | | | | | | | | | |

### Example 31

### Test of the Ex Vivo Effect of the Lyophilized Powder Injection of the Compound of (I) on Mice

Control: 6 BDF-1 mice, male, administer to each mouse 1×10⁸ L₁₂₁₀, a kind of lymph leukemia cell, by intraperitoneal (i.p.) injection, (without the compound of formula (I) treatment); results are shown in FIG. 11.

Ex vivo test group: 6 BDF-1 mice, male, administer to each mouse 1×10⁸ L₁₂₁₀ cells by i.p. injection, wherein the L₁₂₁₀ were treated with the compound of formula (I). The method of treating cells ex vivo: The concentration of the lyophilized powder injection of the compound of formula (I) is 7.5 mg/ml. It was diluted to 150 ug/ml with saline solution. A volume of 67 ul of the diluted injection solution is mixed with 1.5 ml L₁₂₁₀ cells and 0.433 ml saline to obtain in each microliter of L₁₂₁₀ cells to contain 5 ug the compound. The mixture is placed at the room temperature for two hours. The results are shown in Table 4 and 5.

### Results and Conclusion:

TABLE 4
Control group which were administered
with non-treated L₁₂₁₀ cells by i.p.

| BDF-1, | Ave. body | duration of | No. of |
|---|---|---|---|
| male | weight | survival | died mice |
| 6 | 20.8 g | 12 days | 3 |
| | | 13 days | 1 |
| | | 15 days | 2 |

TABLE 5
ex vivo test group which were administered with compound
of formula (I)-treated L1210 cells by i.p.

| BDF-1, | Ave. body | duration of | |
|---|---|---|---|
| male | weight | survival | others |
| 6 | 20.9/each | still survived | no symptom |
| | | after 60 days | |

The results show that the compound of formula (I)—treated L₁₂₁₀ has been killed completely ex vivo and these L₁₂₁₀ cells could not cause cancer in mice.

### Example 32

### The Curable Effect of the Lyophilized Powder Injection of the Compound Of Formula (I) on Mice Transplanted with L₁₂₁₀, a Kind of Lymph Leukemia Cells

Aims: To observe the effect of prolonging the survival of the compound of formula (I) on mice transplanted with L₁₂₁₀, a kind of mouse lymph leukemia cells.

Methods: 18 BDF-1 mice, male. L₁₂₁₀ (mouse lymph leukemia cells) were administered by i.p. injection, at the dose of 1×10⁵ per mouse. Mice were divided into three groups randomly, with six animals per group. The test group was administered with the drug on day 1 and day 6 by subcutaneous (s.c.) injection. An equal volume of saline was administered in control animals.

Dosage: Test group 1: 50 mg/kg the lyophilized powder injection of the compound of formula (I) was administered by s.c. injection,
Test group 2: 25 mg/kg the lyophilized powder injection of the compound of formula (I) was administered by s.c. injection,
Test group 3: 12.5 mg/kg the lyophilized powder injection of the compound of formula (I) was administered by s.c. injection.

Results and conclusion: The effect of prolonging the survival of the compound (I) on mice transplanted with L₁₂₁₀ is shown in table 6. The results indicate that 50 mg/kg lyophilized powder injection of the compound of formula (I) could prolong the survival of the mice transplanted with L₁₂₁₀ significantly. The rate of the prolonging effect is 87%.

TABLE 6
The effect of prolonging the survival of the compound
of formula (I) on mice transplanted with L₁₂₁₀,
a kind of mouse lymph leukemia cells.

| | | method of | duration | prolonging | rate of |
|---|---|---|---|---|---|
| | (dosage | admini- | of survival | days | prolonging |
| Group | mg/kg) | stration | (Ave) | (Day) | (%) |
| Control | 0. d 1, d 6 | s.c. | 15 | — | — |
| Test gp. 1 | 50. d 1, d 6 | s.c. | 28 | 13 | 87% |
| Test gp. 2 | 25. d 1, d 6 | s.c. | 22 | 7 | 47% |
| Test gp. 3 | 12.5 d 1, d 6 | s.c. | 20 | 5 | 33% |

### Example 33

### The Study of Antiviral Effect of the Compound of Formula (I) In Vitro

The study of antivirus effect of the compound of formula (I) in vitro is described as follows.

### Virus: Coxsakie B₃ Virus

Treatment of the sample: The sample was first dissolved in DMSO to appropriate concentration. The sample solutions were then diluted the samples in culture medium solution to obtain eight concentrations.

### Drug as Positive Control: Ribavirin (RBV)

Methods: The Veo cells were cultured in 96-well plates. The cells were infected with Coxsakie B₃ virus for 2 hours after 24 hours of incubation. The solution containing the virus was then discarded. The samples were then added into the wells at the concentrations with the appropriate controls. The cell pathological extent (CPE) was observed 36 hours later. The half inhibitory concentration (IC₅₀) was calculated by using the method of Reed-Muench.

The results are shown in Table 7.

TABLE 7
The study of antivirus effect of the compound of formula(I)

| Sample | TC₅₀ (μg/ml) | Initial con. | IC₅₀ (μg/ml) | SI |
|---|---|---|---|---|
| Compound (I) | 24.7 | 1000 | 8.56 | 2.88 |
| RBV | >1000 | 1000 | 447.8 | >2.23 |

(1) “-” means that the sample has no anti Coxsakie B₃ virus effect at the maximal dose with no toxicity.
(2) TC₅₀ indicates the half toxic concentration; IC₅₀ indicates the half inhibitory concentration for the virus; SI indicates the selective index, calculated as the follow: SI=TC₅₀/IC₅₀

From the table, it can be concluded that the inventive compound has an anti Coxsakie B₃ virus effect.

### Example 34

### The Study of Antifungal Effect of the Compound of (I) In Vitro

The study of antifungal effect of the compound of formula (I) in vitro is described as follows.

Aims: To observe the germicidal effect of the compound of formula (I) on T. rubrum, T. mentagrophytes, C. albicans and B. subtilis.

Method: The method of MTT (3-[4,5-dimethyl(thiazol-2-yl)-3,5-diphery] tetradium bromide) assay.

Preparation of the Suspension: the Sporular Suspension Solution was Prepared by Using the medium to obtain a concentration of 2×10³ spores/ml.

Concentration of the samples were: (1) 2 mg/ml; (2) 1 mg/ml; (3) 0.5 mg/ml; (4) 0.25 mg/ml;

Volume of the sample: 50 ul/well.

### Results and Conclusion:

The compound of formula (I) has significant germicidal effect on T. rubrum, T. mentagrophytes, C. albicans and B. subtilis. The results are shown in Table 8. The results indicate that the antifungal effects reach to 99.9% by using ≧50 μg/well compound of formula (I).

TABLE 8
The germicidal effect of compound of formula (I) on
T. rubrum, T. mentagrophytes, C. albicans and B. subtilis.
Gemicidal effect (killing rate)

| | concentration | | | |
|---|---|---|---|---|
| strain | 100 μg/ml | 50 μg/ml | 25 μg/ml | 12.5 μg/ml |
| T. rubrum | 100% | 99% | 80% | 58% |
| T. mentagrophytes | 100% | 99.5% | 82% | 60% |
| C. albicans | 99.5% | 99% | 75% | 50% |
| B. subtilis. | 100% | 99% | 85% | 65% |

### Example 35

### Preparation of Pharmaceutical Compound

A person skilled in the art would be able to prepare a full range of pharmaceutical preparations comprising the compound of formula (I).

The compound of formula (I), and the pharmaceutically acceptable salts and solvates thereof (hereinafter “the active compounds”), may be administered through oral, parenteral, topical, or rectal routes in the treatment or prevention of cancer, or against bacterial or fungal infections.

In general, these compounds are most desirably administered in dosages ranging from about 0.2 mg per kg body weight per day (mg/kg/day) to about 200 mg/kg/day in single or divided doses (i.e., from 1 to 4 doses per day), although variations will necessarily occur depending upon the species, weight and condition of the subject being treated and the particular route of administration chosen. However, a dosage level that is in the range of about 4 mg/kg/day to about 50 mg/kg/day is most desirably employed.

Variations may nevertheless occur depending upon the species of mammal, fish or bird being treated and its individual response to medicament, as well as on the type of pharmaceutical formulation chosen and the time period and interval at which such administration is carried out. In some instances, dosage levels below the lower limit of the afore range may be more than adequate, while in other cases still larger doses may be employed without causing any harmful side effects, provided that such larger doses are first divided into several small doses for administration throughout the day.

The active compounds may be administered alone or in combination with pharmaceutically acceptable carriers or diluents by the routes previously indicated, and such administration may be carried out in single or multiple doses. More particularly, the active compounds may be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like.

Such carriers include solid diluents or fillers, sterile aqueous media and various non-toxic organic solvents, etc. Moreover, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols.

When aqueous suspensions and/or elixirs are desired for oral administration, the active compound may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of an active compound in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques will known to those skilled in the art.

Additionally, it is also possible to administer the active compounds of the present invention topically and this may be done by way of creams, jellies, gels, pastes, patches, ointments and the like, in accordance with standard pharmaceutical practice.

For administration to animals other than humans, such as cattle or domestic animals, the active compounds may be administered in the feed of the animals or orally as a drench composition.

The active compounds may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active compounds may also be coupled with soluble polymers as targetable drug carriers. Such polymers can include polyvinylpyrrolidone, pyran copolymer, polyhydroxypropylmethacrylamide phenyl, polyhydroxyethylaspartamide-phenol, or polyethyleneoxide-polylysine substituted with palmitoylresidues. Furthermore, the active compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross-linked or amphipathic block copolymers of hydrogels.

The compound of formula (I) may be packaged and sold as a kit of parts, the kit comprising the compound, instructions for use and suitable packaging.

While the compound of formula (I) may be used to treat animals, it is envisage that its bioactive properties may also be used to treat bacterial and fungal infections in plants as well.

1. A compound represented by the following formula (I):

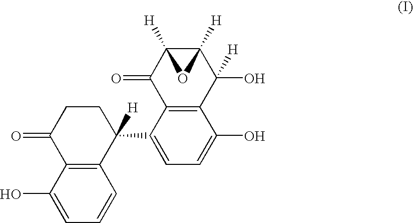

2. An isolated microorganism named as Alternaria alternata var. monosporus with the number of ST-026-R CGMCC No. 0899, wherein the microorganism is suitable for producing the compound of formula (I) of claim 1.

3. The isolated microorganism according to claim 2, wherein the microorganism is cultured under conditions for producing a compound represented by formula (I):

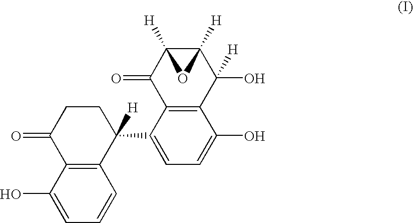

4. The microorganism according to claim 2, wherein the microorganism is cultured in a culture medium for producing a compound represented by formula (I):

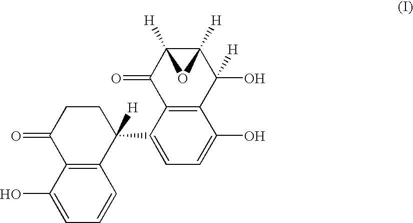

5. A culture comprising the microorganism according to claim 2.

6. A method for producing the compound of formula (I) according to claim 1, comprising culturing the microorganism named as Alternaria alternata var. monosporus with the number of ST-026-R CGMCC No-0899 in a culture medium for producing the compound of formula (I).

7. The method according to claim 6, further comprising the step of collecting and/or purifying the compound of formula (I).

8. A method for producing the compound of formula (I) of claim 1, comprising culturing the microorganism named as Alternaria alternata var. monosporus with the number of ST-026-R CGMCC No. 0899 in a culture medium to produce the compound of formula (I) in the microorganism and/or in the culture medium, and collecting and purifying the compound of formula (I) from the microorganism and/or the culture medium.

9. The method according to claim 6, wherein the culture medium comprises: at least one of the following: glucose, sucrose, maltose, fructose, glycerol, starch, lactose, and galactose, any conventional material comprising a carbon source suitable for the growth of the microorganism, powdered peanut, powdered soybean, corn soup, corn steep solid, corn extract, yeast powder, peptone, beef extract, yeast extract, ammonium nitrate, ammonium chloride, powdered peanut, powdered soybean, a nitrogen source suitable for the growth of the microorganism.

10. The method according to claim 9, wherein the ratio of the at least one carbon source and the at least one nitrogen source is 150:1˜40:1.

11. The method according to claim 6, wherein the culture medium further comprises at least one of the following: phosphate, magnesium salt, ferric salt, sodium salt, a conventional inorganic and/or organic salt suitable for the growth of the microorganism, boric acid, potassium iodide, cobalt bichloride, zinc sulfate, manganese sulfate, at least a trace element, and a conventional material comprising trace elements suitable for the growth of the microorganism.

12. The method according to claim 6, wherein the culture medium further comprises at least one of the following: methyl jasmine, arachidonic acid, ammonium citrate, cerous ammonium nitrate, potassium permanganate, pyruvic acid, coumarinic acid, vanadium sulfate, α-naphthyl-acetic acid, 6-benzyl aminopurine, silver nitrate, cinnamic acid, a conventional inducer suitable for the growth of the microorganism, phenylpropyl amino acid, benzamine, sodium benzoate, sodium acetate, acetamide, propanamide, carbonic acid, ammonium acetate, and a conventional precursor suitable for the growth of the microorganism.

13. The method according to claim 6, wherein the culture medium further comprises at least one inducer.

14. The method according to claim 13, wherein the at least one inducer is selected from the group consisting of methyl jasmine, arachidonic acid, ammonium citrate, cerous ammonium nitrate, potassium permanganate, pyruvic acid, coumarinic acid, vanadium sulfate, α-naphthyl-acetic acid, 6-benzyl aminopurine, silver nitrate, cinnamic acid, and one or more; and/or phenylpropyl amino acid, benzamine, sodium benzoate, sodium acetate, acetamide, propanamide, carbonic acid, and ammonium acetate.

15. The method according to claim 13, wherein the inducer is added while inoculate with the concentration, based on the medium, of 0.005%˜0.1%, and further a precursor is added as the growth of the microorganism enters into the logarithmic phase, at a concentration, based on the medium, of 0.005%˜0.1%.

16. The method according to claim 6, wherein the culture medium further comprises at least one precursor.

17. The method according to claim 16, wherein the at least one precursor is selected from the group consisting of phenylpropyl amino acid, benzamine, sodium benzoate, sodium acetate, acetamide, propanamide, carbonic acid and ammonium acetate.

18. The method according to claim 6, wherein the culture medium comprises at least one of the following: one or more materials providing a carbon source selected from the group consisting of glucose, sucrose, maltose, fructose, glycerol, starch, lactose and galactose; one or more materials providing a nitrogen source selected from the group consisting of the powdered peanut, powdered soybean, corn extract, powdered yeast, peptone, beef extract, yeast extract, ammonium nitrate, and ammonium chloride; one or more inducers selected from the group consisting of methyl jasmine, arachidonic acid, ammonium citrate, cerous ammonium nitrate, potassium permanganate, pyruvic acid, coumarinic acid, vanadium sulfate, α-naphthyl-acetic acid, 6-benzyl aminopurine, silver nitrate and cinnamic acid; and one or more precursors selected from the group consisting of phenylpropyl amino acid, benzamine, sodium benzoate, sodium acetate, acetamide, propanamide, carbonic acid and ammonium acetate.

19. The method of claim 6, wherein the microorganism is cultured in an aerobic condition at temperature of 23˜29° C., wherein the initial pH of the fermentation is 5.5˜11.0; the pH is adjusted to 6.0˜7.5 in the metaphase and anaphase.

20. The method according to claim 7, wherein the collecting and purifying the compound of formula (I) comprises the following steps of:
(a) isolating the cultured microorganism and supernatant obtained from the culture medium;
(b) extracting the cultured microorganism with a first organic solution to obtain a first organic extract, and extracting the supernatant with a second organic solution to obtain a second organic extract, and drying the first and second organic extracts to obtain a product;
(c) purifying the product obtained from the step (b) by at least one chromatography process and at least one crystallization process to obtain a substantially purified compound of formula (I).

21. The method according to claim 20 wherein the at least chromatography process comprises the following conditions:
at least one chromatographic column is used;
stationary phase in the column of the chromatography comprises silica gel or alumina; and
the mobile phase comprises at least one of the following solvent systems selected from the group consisting of ethane/ethyl acetate; methanol/methylene chloride; ethanol/methylene chloride;
and acetone/methylene chloride.

22. The method according to claim 20, wherein the at least one crystallization process comprises:
dissolving the product in an alcohol solution,
adding water;
reducing temperature to 4˜10° C. for 1˜12 hours to separate out the compound of formula (I) in crystallized form;
filtering the compound; and
drying the compound
wherein the at least one crystallization process may be repeated to obtain substantially purified compound of formula (I).

23. The method according to claim 20, wherein the at least one crystallization process comprises:
dissolving the products in ethyl acetate solution;
adding petroleum ether;
cooling to 4˜10° C. to crystallize the compound of formula (I);
filtering the compound; and
drying the compound
wherein the at least one crystallization process may be repeated to obtain substantially purified compound of formula (I).

24. The method of claim 20, wherein before the step (a), the pH of the culture solution is adjusted to pH 2˜9.

25. The method according to claim 20, wherein the microorganism is dried and crushed before step (b).

26-27. (canceled)

28. An anticancer medicament comprising the compound of formula (I) of claim 1.

29. An antifungal medicament comprising the compound of formula (I) of claim 1.

30. An antiviral medicament comprising the compound of formula (I) of claim 1.

31. (canceled)

32. A pharmaceutical composition comprising the compound of formula (I) of claim 1.

33. The pharmaceutical composition according to claim 32, wherein the composition further comprises a pharmaceutically acceptable excipient, diluent and/or carrier.

34. (canceled)

35. A kit of parts for treating a disease comprising the compound according to claim 1 and optionally information related to the use of the compound.

36. A method of treating cancer comprising administering to an animal an effective amount of the compound of formula (I) of claim 1.

37. A method of treating a fungal infection comprising applying to an animal an effective amount of the compound of formula (I) of claim 1.

38. A method of treating a viral infection comprising administering to an animal an effective amount of the compound of formula (I) of claim 1.

39. The method according to claim 36, wherein the animal is a mammal.

40. The method according to claim 39, wherein the mammal is human.
